(19) United States
(12) Patent Application Publication
(10) Pub. No.: US 2025/0349424 A1
(43) Pub. Date: Nov. 13, 2025

(54) PROLONGED AIR LEAK PERCEPTION

(71) Applicant: Texas Medical Center, Houston, TX (US)

(72) Inventors: William Cohn, Bellaire, TX (US); Steven Nguyen, Cypress, TX (US); Jorge Salazar, Houston, TX (US); Matthew Kuhn, Houston, TX (US); Haowei Ni, Raritan, NJ (US); Rajitha Aluru, Austin, TX (US)

(21) Appl. No.: 18/860,528

(22) PCT Filed: Apr. 20, 2023
(86) PCT No.: PCT/IB2023/054040

§ 371 Date: Oct. 25, 2024

Related U.S. Application Data
(60) Provisional application No. 63/363,631, filed on Apr. 26, 2022.

(51) Int. Cl. G16H 50/20; A61B 5/00; A61B 5/08; G16H 10/60; G16H 50/30; G16H 50/70

(52) U.S. Cl. CPC G16H 50/20; A61B 5/08; A61B 5/7275; G16H 10/60; G16H 50/30; G16H 50/70

(57) ABSTRACT

A device may include a processor. The processor may be configured to receive data during a surgery. The processor may receive data representative of a patient's intraoperative air exchange. The data may represent air exchange for a patient's breath cycle. For example, data may include any of ventilator inlet flow rate, ventilator inlet pressure, ventilator output pressure, chest tube flow rate, or chest tube pressure. The processor may also receive data representative of a surgical parameter other than one related to air exchange. For example, this data may include any of patient medical record data, intraoperative reporting data, surgical procedure data, or the like. The processor may be configured to receive an updated machine learning model from a cloud resource. And the processor may output, during the surgery, information indicative of prolonged-air-leak-likelihood based on data.

### CROSS-REFERENCE TO RELATED APPLICATIONS

This application claims priority to, and the benefit of, under 35 U.S.C. § 119(e) of U.S. Provisional Appl. No. 63/363,631, filed Apr. 26, 2022, which is incorporated by reference herein in its entirety.

### BACKGROUND

A prolonged air leak occurs when air escapes a patient's lungs into the chest cavity for an unacceptable length of time, often 5-7 days. Such an air leak may occur after lung surgery, with a traumatic injury, a lung biopsy, or the like. Moreover, a prolonged air leak may occur in a significant number of patients after pulmonary resection. And this complication may be associated with increased time in hospital and major postoperative morbidity (e.g., lobar collapse, nosocomial pneumonia, and pleural empyema). All of which drives higher healthcare costs and poorer patient outcomes.

Although preventive strategies have been investigated to mitigate the risk of prolonged air leak, including, for example, surgical techniques, sealants, and buttressing materials, none have proved definitively effective.

### SUMMARY

A device may include a processor. The processor may be configured to receive data during a surgery. The processor may receive data representative of a patient's intraoperative air exchange. The data may represent air exchange for a patient's breath cycle. For example, data may include any of ventilator inlet flow rate, ventilator inlet pressure, ventilator output pressure, chest tube flow rate, or chest tube pressure. The processor may also receive data representative of a surgical parameter other than one related to air exchange. For example, this data may include any of patient medical record data, intraoperative reporting data, surgical procedure data, or the like. The processor may be configured to receive an updated machine learning model from a cloud resource. And the processor may output, during the surgery, information indicative of prolonged-air-leak-likelihood based on data.

### BRIEF DESCRIPTION OF THE DRAWINGS

FIG. 1 is depiction of a computerized tomography (CT) scan of a patient's chest cavity.

FIG. 2 is depiction of a lung resection with chest tube.

FIG. 3 illustrates an example signal for a lung that does not develop a prolonged air leak.

FIG. 4 illustrates an example signal for a lung that does develop a prolonged air leak.

FIG. 5 is a graph of example signals.

FIG. 6 is a graph of an example signal comprised of two time series.

FIG. 7 is a block diagram illustrating an example system for determining the likelihood of developing a prolonged air leak.

FIG. 8 is a block diagram of an example device for collecting signals and for determining the likelihood of developing a prolonged air leak.

FIG. 9 is a flow diagram of an example process for collecting signals and for determining the likelihood of developing a prolonged air leak.

FIG. 10 is an illustration of an example device for collecting signals and for determining the likelihood of developing a prolonged air leak.

FIG. 11 is a block diagram of an example device for collecting signals and for determining the likelihood of developing a prolonged air leak.

FIG. 12 is a flow diagram of an example process for collecting signals and for determining the likelihood of developing a prolonged air leak.

FIG. 13 is an illustration of an example device for collecting signals and for determining the likelihood of developing a prolonged air leak.

FIG. 14 is a block diagram of a system incorporating an example plurality of surgical computing devices.

FIG. 15 is a block diagram illustrating an example system for determining the likelihood of developing a prolonged air leak.

FIG. 16 is a flow diagram of an example process for collecting signals and for determining the likelihood of developing a prolonged air leak.

### DETAILED DESCRIPTION

FIG. 1 is depiction of a computerized tomography (CT) scan of a patient's chest (e.g., thoracic) cavity 100. The chest cavity 100 contains the lungs 102, the heart 104, and other organs such as major blood vessels. The surface of the lung 106 and the inside of the chest wall 108 is covered by a plural membrane. In a healthy patient, lungs 102 may be fully inflated within the cavity 100 because the pressure inside the lungs 102 is generally higher than the pressure inside the pleural space 110.

Here however, the CT scan shows a pneumothorax 112 on the patient's left side (the right side of the image). In certain conditions, such as damage to the chest wall 108 and or damage to one or both lungs 102 for example, a pneumothorax 112 may develop. Air may enter the pleural cavity 110. And intrapleural pressure may increase. Such an increase in pressure may result in normalizing the pressure difference between the lung pressure and the intrapleural pressure. This may cause one or both lungs 102 to deflate—a life threatening condition for the patient.

In thoracic surgery, particularly where some portion of the lung is removed, a typical consequence is for some air to leak from the lungs 102 and enter the pleural space 110. These air leaks typically diminish as any surgical wounds to the lungs 102 heal. However, when such air leaks do not diminish in a suitable amount of time, they are considered a prolonged air leak (PAL) and the consequences for the patient may be significant. So, a chest tube may be used after surgery to allow the leaked air to escape the pleural cavity 110.

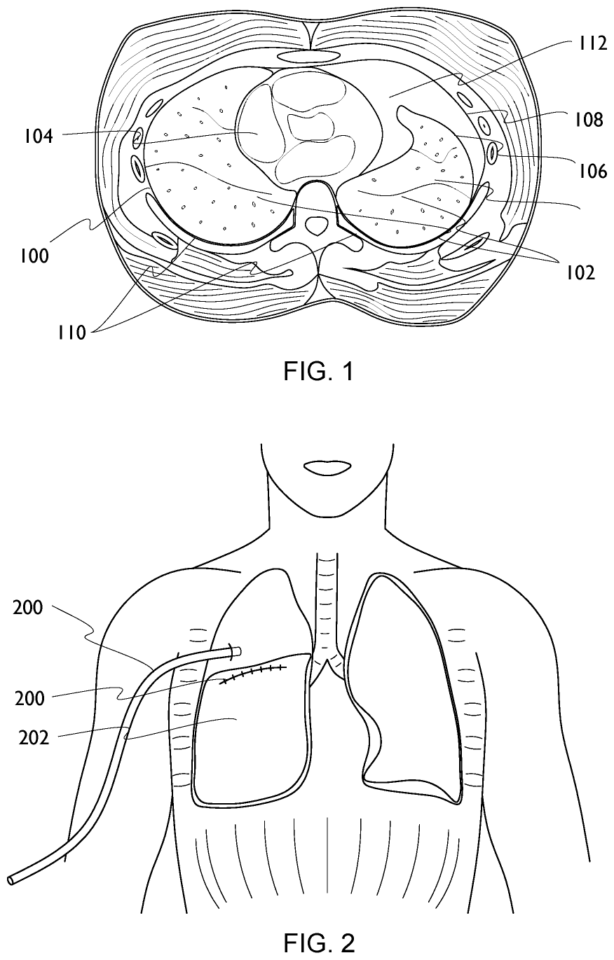

FIG. 2 is depiction of a lung resection with a chest tube 200. A lung resection surgery may involve removing an entire lung, e.g., a pneumonectomy; a lobe of a lung, e.g., a lobectomy; or a portion of a lobe, e.g., a segmental or wedge resection. The remaining portion of lung 202 may have a wound from the resection that is surgically closed. Such a closed wound 204 may be closed with any appropriate surgical technique, such as sutures, surgical glue, staples, and/or the like, alone or in combination.

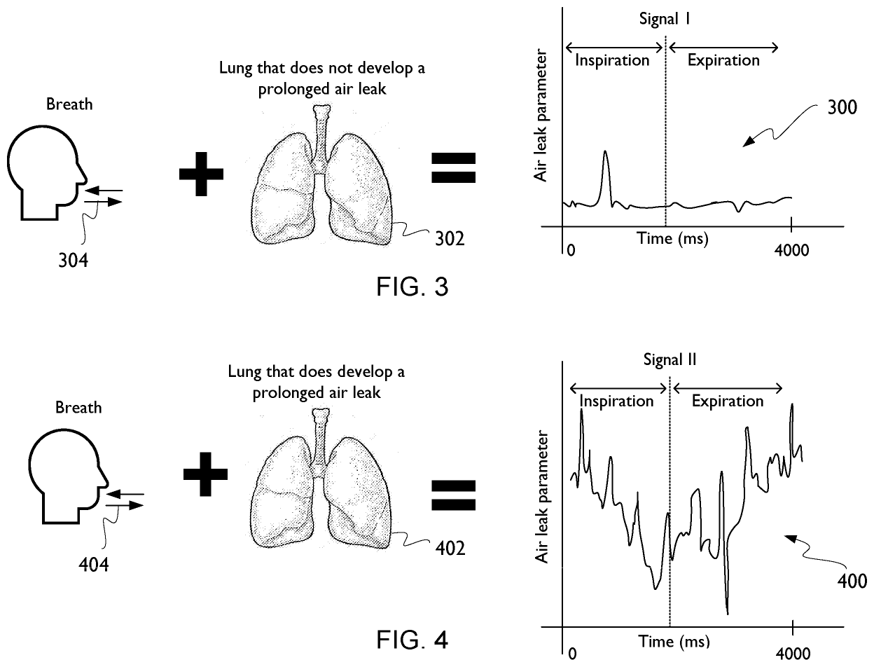

Because it is common for there be an air leak while the lung 202 and the closed wound 204 heals, a chest tube 200 is routinely installed at the completion of a lung resection operation. The chest tube 200 allows for air leakage from the lung into the pleural cavity to escape the body. The chest tube 200 may allow for fluid drainage as well. In some instances, more than one chest tube 200 may be used. The chest tube 200 may be connected to a drainage system, such as an underwater drainage system. The drainage system may be connected to wall suction. And when leakage of air and/or fluid drainage has ceased or has reached an acceptably low amount, the chest tube may be removed. And when it does not, the patient has a prolonged air leak, which threatens the patient's recovery and may require further treatment and even subsequent surgical intervention. FIGS. 3 and 4 illustrate an approach to predicting whether an air leak will heal or will persist over multiple days and become a prolonged air leak. Such a prediction, especially if available during surgery, may enable the surgeon to take additional steps to mitigate or prevent this dangerous complication.

FIG. 3 illustrates an example signal 300 for a lung that does not develop a prolonged air leak. Consider the lung 302 as a system and the breath 304 an input to that system. A granular measurement of an air leak parameter may serve as a signal representative of the system. For example, the air leak parameter may include a granular measurement of the air mass flow through a chest tube. This parameter may be measured and/or sampled rapidly over the course of a full breath, for example. The resulting signal 300 may include a time series of that parameter. A portion of which may be associated with inspiration (e.g., in breath), and another portion of which may be associated with expiration (e.g., out breath). The breath 304 itself may be provided by the patient, a ventilator, or the like. And the measurements may be made, for example, after a lung resection but before closing the patient and concluding the surgery.

Here, the leak rate measured as the airflow through a chest tube is sampled at a significantly high frequency to capture the variations of the rate of airflow through the chest tube over the course of a breath 304. These variations over a short duration (e.g., about 4000 ms) can provide detailed insight into the health of the lung 302 and can be used to predict the likelihood of a prolonged air leak. In this example signal 300, a breath cycle 304 though the lung 302 generates a signal 300 that is relatively low and flat, for example. An analysis of such a signal may predict the patient not developing a prolonged air leak after the lung resection surgery.

By way of contrast, FIG. 4 illustrates an example signal 400 for a lung 402 that does develop a prolonged air leak. This signal 400 is remarkably different from the signal 300 generated by the lung that does not develop a prolonged air leak. Here, the example signal 400 generally oscillates, exhibiting a correlation with the inspiration and expiration of the breath 404. The example signal 400 contains a number of defined peaks and valleys. Such peaks and valleys may be characterized by their number, frequency, magnitude, and the like. The overall flowrate is generally higher. The air leak characteristics of the lung 402 may be represented by the nature of these intra-breath variations. Such variations may become detectable with the appropriate granularity of measurement (e.g., sampling rate). And an analysis of such a signal 400 may predict the patient not developing a prolonged air leak after the lung resection surgery.

The air leak parameter illustrated in FIGS. 3 and 4 may include one or more measurable parameters associated with air moving in, out, and/or through the lungs and/or chest cavity. For example, the air leak parameter may include one or more of ventilator inlet flow rate, ventilator inlet pressure, ventilator output flow rate, ventilator output pressure, chest tube flow rate, chest tube pressure, and/or the like. The signal 300, 400 may include a time series of one or more such parameters, for example.

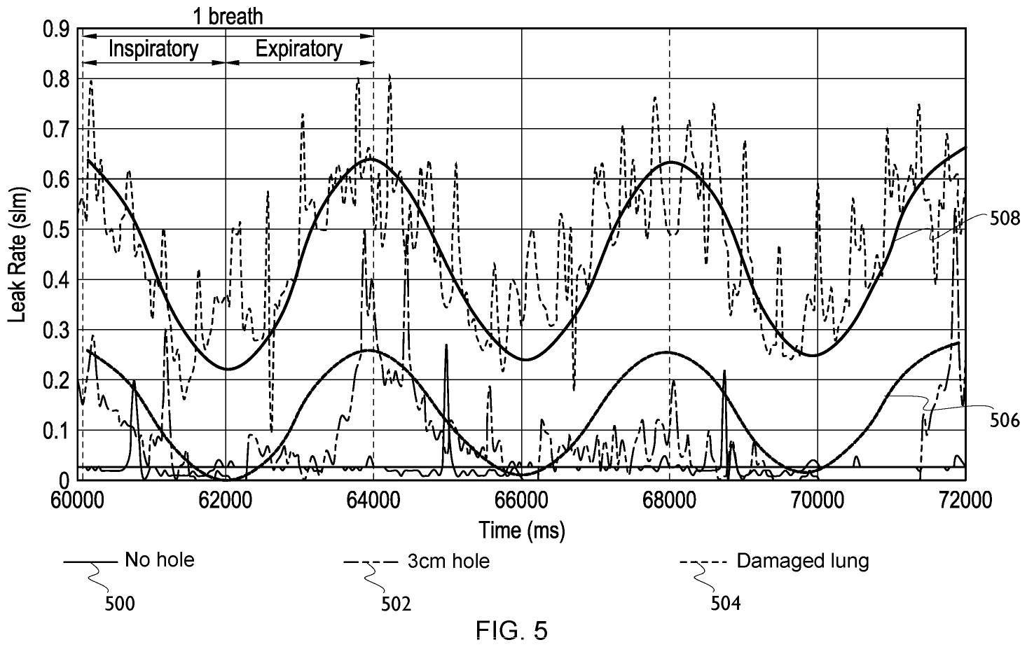

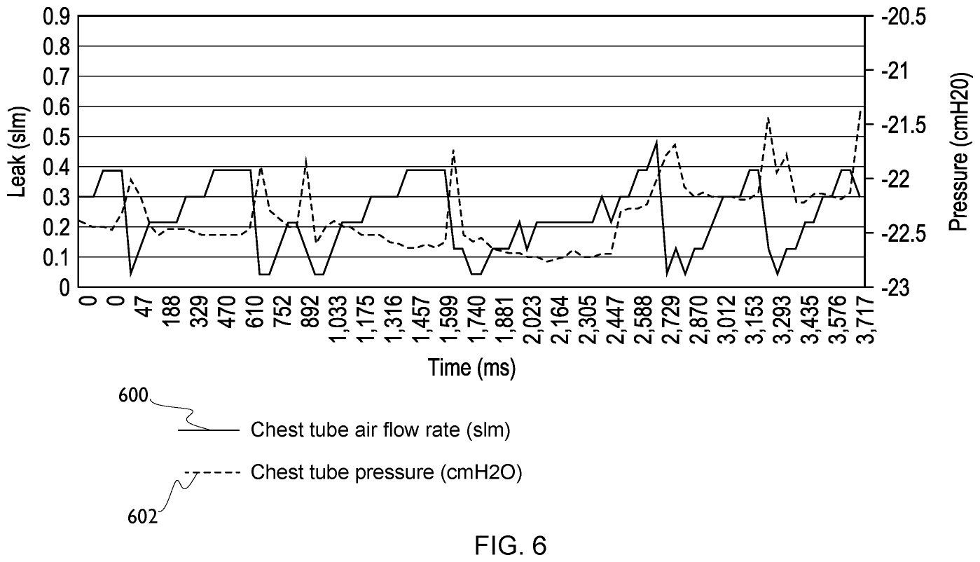

For example, FIGS. 5 and 6 illustrate a number of example signals that may be used to assess the likelihood of a patient developing a prolonged air leak. FIG. 5 is a graph of example signals. It is a plot of chest tube leak rate over time of three breath cycles for lungs with different physical characteristics. The first signal 500 is that of an example healthy lung with no holes. The second signal 502 is that of a lung with a 3 cm hole in a bottom lobe. And the third signal 504 is that of a more significantly damaged lung.

The first signal 500 shows a generally flat profile with a characteristic peak during the inspiratory portion of the breath cycle. The second signal 502 exhibits certain peaks and valleys, a general oscillation with the breath, and a relatively low overall magnitude. The general oscillation is shown with an overlaid line 506. The third signal 504 is like the second signal 502 but with a greater overall leak rate and oscillation magnitude (as shown with overlaid line 508).

FIG. 6 is a graph of an example signal comprised of two time series. Here, the first time series 600 is that of chest tube airflow rate. And the second time series 602 is that of chest tube pressure. Such an example signal has two dimensions one for flow and the other for pressure. Here, not only do individual time series contain information predictive of prolonged air leaks, but the interrelationship between the two time series does as well. An example signal may include one or more time series. For example, the time series may be time correlated. For example, the time series may represent a concurrent duration of time. Here the correlations and/or relationships between the pressure curve and the flow rate curve may include including number of peaks, the variance between peaks, the total area under the curve, and the like.

In an example, the signal and/or one or more characteristics of the signal may be used to assess the likelihood of a prolonged air leak. For example, the signal and/or one or more characteristics of the signal may be used to determine that a present lung is in a condition that a prolonged air leak is likely or not likely. Signal characteristics may include the number of peaks in the flow and/or pressure curves during inspiration and/or expiration, the total leak per breath cycle (area under the flow curve), the difference between two different peaks of the flow and/or pressure curves, and the like.

As depicted in FIGS. 5 and 6, the measured parameters are sampled at a relatively high rate. A digital chest tubes drainage system may provide a static or “snapshot” view of flow. And health care professionals may use that static view over the course of hours and/or days to determine whether a patient is safe for chest tube removal. However, a static or “snapshot” view fails to capture the intra-breath variations indicative of lung leakage and that can be used to train a predictive model and ultimately be used to predict the likelihood of developing a prolonged air leak. The devices, systems, and methods disclosed herein may employ sampling of measured parameters at a higher rate, one suitable for capturing these indicative intra-breath variations.

The selection of suitability of sampling rate may be discerned in accordance with the Nyquist rate associated with the analog parameter being measured. For example, the chest tube rate and pressure and or parameters associated with air exchange may represent a continuous function. That continuous function may be characterized by a frequency range. Selecting a rate of the sampler to be greater than twice the highest relevant frequency of the continuous function, the resultant discrete time sequence may be free of distortion and may be used to preserve and/or recreate the information present in the original relevant signal at a desired fidelity. In an example, the frequency range or spectrum of the continuous signal associated with airflow exchange may be determined for a population, and then an appropriate Nyquist rate for the sampler may be selected for use with that parameter and/or population. In an example, the sampling rate may include a sampling period of one second, 500 milliseconds, 100 milliseconds, 50 milliseconds, or faster.

Signals and/or their characteristics may be analyzed for a population. One or more signal profiles may be developed for the population. Such profiles may be associated with various degrees of likelihood of developing a prolonged air leak. For example, signals and/or their characteristics may be analyzed for a population of resection surgeries associated with the non-development of a prolonged air leak. For example, signals and/or their characteristics may be analyzed for a population of resection surgeries associated with the development of a prolonged air leak. A subsequent signal and/or signal characteristics may be compared one or more profiles and/or other signals to assess whether the particular subsequent signal is more like those of the population of resection surgeries associated with the non-development of a prolonged air leak or the population of resection surgeries associated with the development of a prolonged air leak.

A computer system may configured to perform an analysis of the signals and/or signal characteristics for a population. The computer system may be configured with an analytics program to assess likelihood. The analytics program may include any process suitable for comparing a signals and/or signal characteristics to one or more populations of signals and/or signal characteristics. The analytics programing may include approaches such as regression analysis, linear regression, nonlinear regression, vector autoregression, and/or machine learning. This computer system may be configured to develop a predictive model, for example.

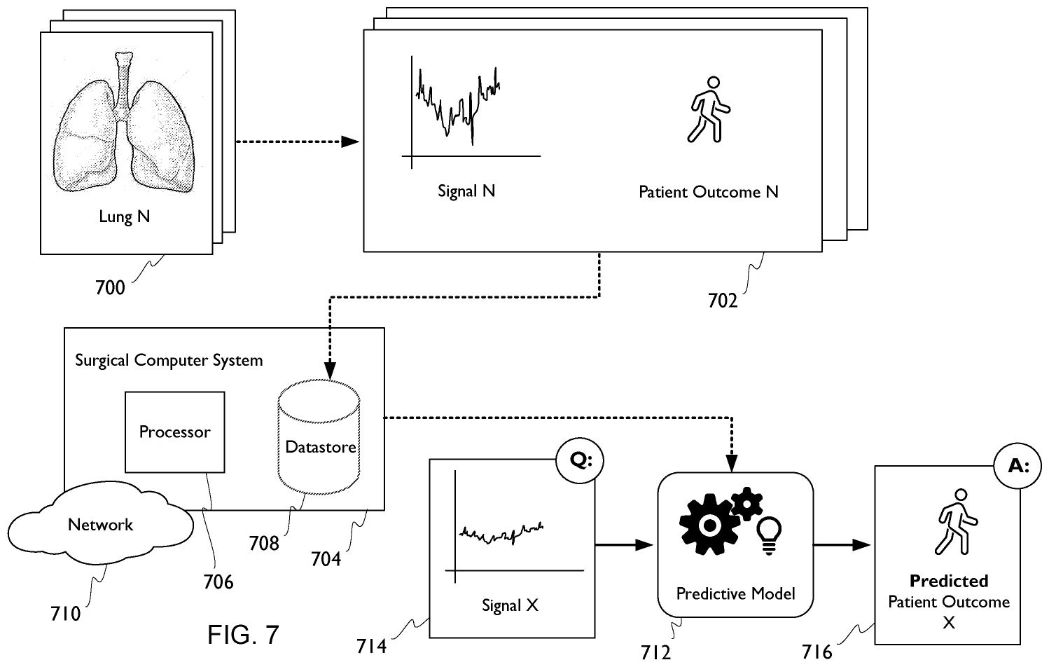

FIG. 7 is a block diagram illustrating an example system for determining the likelihood of developing a prolonged air leak. Surgical data 700 may be captured from one or more resection surgeries. For example, surgical data 700 may be captured from a large number of resection surgeries. For example, the data may be collected from at least a hundred resection surgeries. The surgical data 700 may comprise one or more signals indicative of the air exchange of the respective patient. The surgical data 700 may be combined with medical record data to generate one or more data elements 702, each element containing at least a signal and/or signal characteristics and a corresponding patient outcome. For example, the patient outcome may include whether the patient developed a prolonged air leak. For example, the patient outcome may include the number of days a leak was present and on which day it reached an acceptable level.

The surgical data 700 and/or the data elements 702 may be stored and processed by a surgical computer system 704. The surgical computer system 704 may include a processor 706 and/or a datastore 708. The surgical computer system 704 may be operable over a computer network 710. The collected data may populate the datastore 708 such that a predictive model 712 may be generated. The processor 706 may be configured to generate the predictive model 712 based on the surgical data 700 and/or the data elements 702 stored in the datastore 708, for example. The predictive model 712 may be generated by traditional data analysis techniques, machine learning, and the like.

The predictive model 712 may be generated by the processor 706 in accordance with any appropriate machine learning technique. For example, the processor 706 may use a supervised learning algorithm. A supervised learning algorithm may create a mathematical model from training a dataset (e.g., training data). The training data may consist of a set of training examples. A training example may include one or more inputs and one or more labeled outputs. Data elements 702 may be used as training data. The labeled output(s) may serve as supervisory feedback. In a mathematical model, a training example may be represented by an array or vector, sometimes called a feature vector. The training data may be represented by row(s) of feature vectors, constituting a matrix. Through iterative optimization of an objective function (e.g., cost function), a supervised learning algorithm may learn a function (e.g., a prediction function) that may be used to predict the output associated with one or more new inputs. A suitably trained prediction function may determine the output for one or more inputs that may not have been a part of the training data. Example algorithms may include linear regression, logistic regression, and neutral network. Example problems solvable by supervised learning algorithms may include classification, regression problems, and the like.

In an example, the processor 706 may use an unsupervised algorithm to develop the predictive model 712. An unsupervised learning algorithm may train on a dataset that may contain inputs and may find a structure in the data. The structure in the data may be similar to a grouping or clustering of data points. As such, the algorithm may learn from training data that may not have been labeled. Instead of responding to supervisory feedback, an unsupervised learning algorithm may identify commonalities in training data and may react based on the presence or absence of such commonalities in each train example. The surgical data 700 may serve as training data. Example algorithms may include Apriori algorithm, K-Means, K-Nearest Neighbors (KNN), K-Medians, and the like. Example problems solvable by unsupervised learning algorithms may include clustering problems, anomaly/outlier detection problems, and the like

In an example, the processor 706 may use a reinforcement learning algorithm to develop the predictive model 712. Reinforcement learning is an area of machine learning that may be concerned with how software agents may take actions in an environment to maximize a notion of cumulative reward. Reinforcement learning algorithms may not assume knowledge of an exact mathematical model of the environment (e.g., represented by Markov decision process (MDP)) and may be used when exact models may not be feasible. Reinforcement learning algorithms may be used in autonomous vehicles or in learning to play a game against a human opponent.

The output of machine learning's training process may be a model for predicting outcome(s) on a new dataset, such as the predictive model 712 being used with the subsequent signal information 714, for example. A linear regression learning algorithm may be a cost function that may minimize the prediction errors of a linear prediction function during the training process by adjusting the coefficients and constants of the linear prediction function. When a minimal may be reached, the linear prediction function with adjusted coefficients may be deemed trained and constitute the model the training process has produced. For example, a neural network (NN) algorithm (e.g., multilayer perceptrons (MLP)) for classification may include a hypothesis function represented by a network of layers of nodes that are assigned with biases and interconnected with weight connections. The hypothesis function may be a non-linear function (e.g., a highly non-linear function) that may include linear functions and logistic functions nested together with the outermost layer consisting of one or more logistic functions. The NN algorithm may include a cost function to minimize classification errors by adjusting the biases and weights through a process of feedforward propagation and backward propagation. When a global minimum may be reached, the optimized hypothesis function with its layers of adjusted biases and weights may be deemed trained and constitute the model the training process has produced.

The processor 706 may be used to perform any elements of the machine learning lifecycle, including stages such as data collection, data preparation, model training, model deployment, post-deployment, and the like.

Data collection may be performed for machine learning as a first stage of the machine learning lifecycle. For example, data collection may include steps such as identifying various data sources, collecting data from the data sources, integrating the data, and the like.

Data preparation may include data preprocessing steps such as data formatting, data cleaning, and data sampling. Data preparation may include data transforming procedures (e.g., after preprocessing), such as scaling and aggregation. For example, the preprocessed data may include data values in a mixture of scales. These values may be scaled up or down, for example, to be between 0 and 1 for model training. For example, the preprocessed data may include data values that carry more meaning when aggregated.

Model training involves applying an appropriate machine learning algorithm to the prepared data. A model may be deemed suitably trained after it has been trained, cross validated, and tested. Accordingly, the dataset from the data preparation stage (e.g., an input dataset) may be divided into a training dataset (e.g., 60% of the input dataset), a validation dataset (e.g., 20% of the input dataset), and a test dataset (e.g., 20% of the input dataset). After the model has been trained on the training dataset, the model may be run against the validation dataset to reduce overfitting. If accuracy of the model were to decrease when run against the validation dataset when accuracy of the model has been increasing, this may indicate a problem of overfitting. The test dataset may be used to test the accuracy of the final model to determine whether it is ready for deployment or more training may be required.

Model deployment may include how the model is used. For example, the model may be deployed as a part of a standalone computer program. The model may be deployed as a part of a larger computing system. In an example, the predictive model 712 may be deployed in a computer system, an embedded system, a surgical computer system (e.g., a surgical hub), a cloud-based system, and the like. For example, the predictive model 712 may be deployed in systems, devices, and methods disclosed herein.

A model may be deployed with model performance parameters(s). Such performance parameters may monitor the model accuracy as it is used for predicating on a dataset in production. For example, such parameters may keep track of false positives and false negatives for a classification model. Such parameters may further store the false positives and false negatives for further processing to improve the model's accuracy.

Post-deployment model updates may be another aspect of the machine learning cycle. For example, a deployed model may be updated as false positives and/or false negatives are predicted on production data. In an example, for a deployed MLP model for classification, as false positives occur, the deployed MLP model may be updated to increase the probably cutoff for predicting a positive to reduce false positives. In an example, for a deployed MLP model for classification, as false negatives occur, the deployed MLP model may be updated to decrease the probably cutoff for predicting a positive to reduce false negatives. In an example, for a deployed MLP model for classification of surgical complications, as both false positives and false negatives occur, the deployed MLP model may be updated to decrease the probably cutoff for predicting a positive to reduce false negatives because it may be less critical to predict a false positive than a false negative.

For example, a deployed model may be updated as more live production data become available as training data. In such case, the deployed model may be further trained, validated, and tested with such additional live production data. In an example, the updated biases and weights of a further-trained MLP model may update the deployed MLP model's biases and weights.

In an example, the predictive model 714 may be generated, validated, and ultimately deployed. For example, a subsequent signal 714 may be input to the predictive model 712 to provide an output 716. The output 716 may include a probability of whether the subsequent signal 714 is associated with the development of a prolonged air leak. The subsequent signal 714 may be one that was collected from surgical data that not part of the surgical data 700 used to generate the predictive model 712. For example, the subsequent signal 714 may include a testing signal in which the patient outcome is known. A testing signal may be used to confirm the accuracy of the predictive model. For example, the subsequent signal 714 may include a new patient signal from a patient in which the outcome is not yet known. A new patient signal may be used such that the output of the predictive model may be used by the surgeon or other health care professional assess the likelihood of the patient developing a prolonged air leak. In this capacity, such a model 712 may enable intraoperative interventions and/or earlier postsurgical interventions.

Such a model 712 confers a substantial clinical and economic benefit to patients, clinicians, healthcare facilities, and the like. For example, by having such information in the operating theater, the surgeon may perform further surgical tasks to address potential leakages that would otherwise been seen as unnecessary. For example, the surgeon may provide extra sealant, sutures, staples, or the like to a wound in the lung. For example, the surgeon may perform further diagnostics associated with lung leakages, such as submerging it in water to find yet-unseen leaks. For example, the surgeon may provide for other surgical mitigating care. Such information regarding the likelihood of developing a prolonged air leak may be particularly helpful during the resection surgery because these additional surgical activities may be completed while the patient is still in the operating theater prior to closing, foregoing the need for a subsequent surgical intervention.

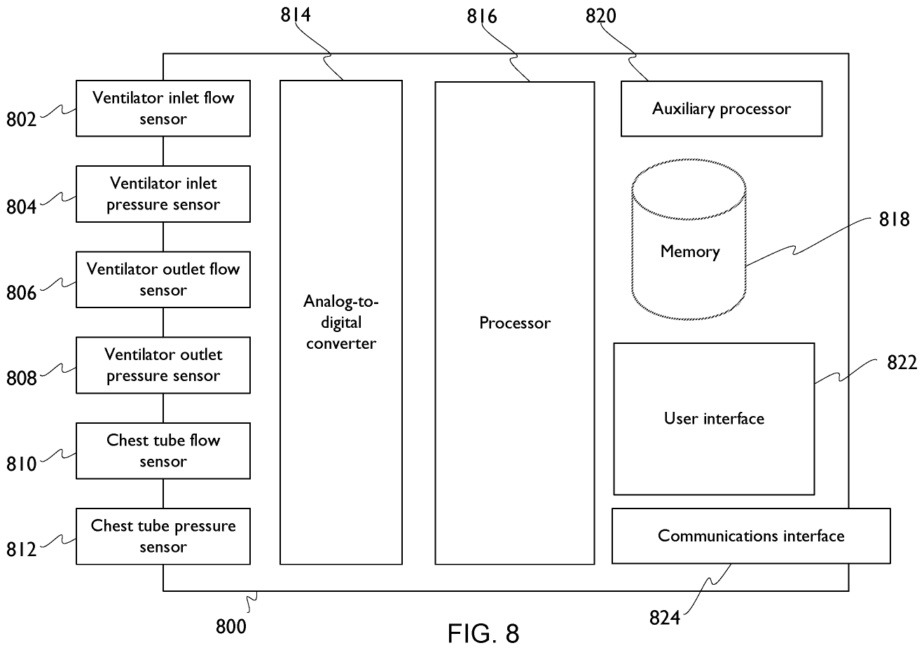

FIG. 8 is a block diagram of an example device 800 for collecting signals and/or for determining the likelihood of developing a prolonged air leak. For example, the device 800 may be suitable for capturing signal information for a population for purposes of training a predictive model. For example, the device 800 may be suitable for capturing subsequent signal information, such as a testing or new patient signal for example, for purposes of considering the output of a predictive model. For example, the device 800 may be used to capture subsequent signals to be input to the predictive model and to provide a predicted patient outcome, such as the likelihood of the patient developing a prolonged air leak, to the surgeon.

The device 800 may include one or more sensors 802, 804, 806, 808, 810, 812. The device 800 may include any sensors suitable for collecting a signal indicative of patient air exchange. For example, the device 800 may include any sensors suitable for collecting a signal indicative of air exchange between a surgical ventilator and a patient. For example, the device 800 may include any sensors suitable for collecting a signal indicative of air leaving the patient via a chest tube. For example, the device 800 may include one or more sensors such as, a ventilator inlet flow sensor 802, a ventilator inlet pressure sensor 804, a ventilator outlet flow sensor 806, a ventilator outlet pressure sensor 808, a chest tube flow sensor 810, a chest tube pressure sensor 812, and the like. Such sensors may be suitable for recording surgical-quality data from a patient. For example, airflow data may be measured with a tolerance of +/−0.4 liters per minute. For example, pressure measurements may be made with a tolerance of +/−2.5 centimeters H2O.

The flow sensors 802, 806, 810 may include any sensor suitable for capturing the flow rate of air. For example, the flow sensors 802, 806, 810 may include sensors or flow meters designed for medical applications. For example, the flow sensors 802, 806, 810 may be used to withstand autoclave procedures. For example, the flow sensors 802, 806, 810 may be packaged for single use and/or for a multiple use. For example, the flow sensors 802, 806, 810 may be designed for medical ventilation or respiratory applications. In an example, the flow sensors 802, 806, 810 may include an analog sensor and/or digital sensor. For example, the flow sensors 802, 806, 810 may include one or more silicone sensor chips. For example, one or more flow sensors 802, 806, 810 may include relevant support circuitry such as an amplifier, integrated A/D converter, EEPROM memory, digital signaling processing circuitry, and interface circuitry, and the like. In an example, one or more flow sensors 802, 806, 810 may include the SFM3400 digital flow meter from Sensirion™.

The pressure sensors 804, 808, 812 may include any sensor suitable for measuring air pressure. The pressure sensors 804, 808, 812 may include any sensor suitable for measuring absolute, gauge, and/or differential air pressures, for example. The pressure sensors 804, 808, 812 may include sensors or pressure meters designed for medical applications. For example, the pressure sensors 804, 808, 812 may be used to withstand autoclave procedures. For example, pressure sensors 804, 808, 812 may be packaged for single use and/or for a multiple use. For example, the pressure sensors 804, 808, 812 may include any pressure sensor suitable for medical applications, such as for air monitors, pneumatic controls, respiratory machines, ventilators, spirometers, and the like. In an example, the pressure sensors 804, 808, 812 may include an analog sensor and/or digital sensor. For example, the pressure sensors 804, 808, 812 may include one or more silicone sensor chips. For example, one or more pressure sensors 804, 808, 812 may include relevant support circuitry such as an amplifier, integrated A/D converter, EEPROM memory, digital signaling processing circuitry, and interface circuitry, and the like. In an example, one or more pressure sensors 804, 808, 812 may include a board mount pressure sensor, such as the board mount pressure sensor from Honeywell™ part number 785-HSCDRRN100MD4A3, for example.

Measurements sensed by the sensors 802, 804, 806, 808, 810, 812 may be converted to digital representation via one or more analog-to-digital converters 814. The analog-to-digital converter 814 may convert an analog representation of the sensor's measurement, such as a voltage, current, or the like, into a digital representation, such as an 8 bit, 16 bit, 24 bit, 32 bit digital value, for example. The analog-to-digital converter 814 may include any architecture and/or form factor suitable for inclusion in a medical device, such as device 800. For example, the analog-to-digital converter 814 may include a converter integrated with one or more sensors themselves. The analog-to-digital converter 814 may include a subcomponent of the processor 816. The analog-to-digital converter 814 may include a standalone electrical component, for example. The analog-to-digital converter 814 may include an individual converter for each sensor, a shared converter, or a combination thereof.

The analog-to-digital converter 814 may convert analog information captured by the one or more sensors 802, 804, 806, 808, 810, 812 to a digital format by sampling the signals received from the sensors at a particular sampling rate. The sampling rate may be any rate suitable for capturing a signal and/or signal characteristics of air exchange of a patient. For example, the sampling rate may be selected to be at least twice the highest relevant frequency for the type of signal being sampled. For example, the sampling rate may be selected as discussed herein. The digital signals from the analog-to-digital converter 814 may represent a time series of data for each of the sensors 802, 804, 806, 808, 810, 812 of the device 800. The captured data, for example the one or more time series of data, may be stored in memory 818 and/or processed by the processor 816.

The processor 816 may include any device suitable for processing such data. For example, the processor 816 may include any device suitable for handling such data, performing numeric operations on such data, storing the data to memory 818, operating a predictive model with the data as input, handling operation of the device 800, and/or the like. The processor 816 may include a general-purpose processor, a microcontroller, an application specific integrated circuit (ASIC), or the like. In an example, the processor may include an Arduino Uno microcontroller, for example.

The memory 818 may include any component suitable for storing such digital data. For example, the memory 818 may include random access memory, read-only memory, volatile memory and/or non-volatile memory. For example, the memory 818 may include a solid-state memory or the like. The memory 818 may be sized and selected to be suitable for the volume and storage speed required in accordance with the sensors 802, 804, 806, 808, 810, 812 and processor 816.

The device 800 may include one or more auxiliary processors 820. An auxiliary processor 820 may include any component, device, system, computing and/or resource and/or access to such component, device, system, and/or computing resource used to provide processing additional to the processing of processor 816, for example. The auxiliary processor 820 may include a Field Programmable Gate Array (FPGA), an application specific integrated circuit (ASIC), an application programming interface (API) to an external processing resource, such as a cloud and/or edge processing resource, and/or the like. In an example, an auxiliary processor may be used to handle the computing requirements of developing and/or implementing a predictive model, such as that discussed herein.

The device 800 may include a user interface 822. The user interface 822 may provide user input mechanisms, such as buttons, touchscreens, and/or access to external user interface devices, such as a keyboard, monitor, and mouse, for example. The user interface 822 may provide a user input mechanism to establish the beginning and/or the end of a signal recording session. The user interface 822 may provide the ability to input certain patient-related data, for example. The user interface 822 may provide a user output mechanism, such as indicator lights, a display, access to external user interface devices, and/or the like. The output mechanism may be used to output all or a portion of the information captured by the sensors 802, 804, 806, 808, 810, 812. The output mechanism may be used to output a summary of the information captured by the sensors 802, 804, 806, 808, 810, 812. The output mechanism may be used to output a result of predictive model processing data captured by the sensors 802, 804, 806, 808, 810, 812. For example, the output mechanism may include a likelihood of a prolonged air leak based one data captured from one or more of the sensors 802, 804, 806, 808, 810, 812. For example, a display may be used for displaying information indicative of prolonged-air-leak-likelihood. In an example, the displayed output information indicative of prolonged-air-leak-likelihood may include a numerical value. In an example, the displayed output information indicative of prolonged-air-leak-likelihood may include a qualitative risk level.

The device 800 may include a communications interface 824 to provide data exchange between the device 800 and one or more other components and/or networks. For example, the communication interface 824 may include a serial interface, a parallel interface, a universal serial bus (USB) interface, and the like. For example, the communication interface 824 may include a network communication interface, such as an Ethernet interface, a WiFi interface, a cellular interface, a 5G interface, and/or the like. The communications interface 824 may provide access to a host computer, for data logging capabilities, for example. The communications interface 824 may provide data exchange between the device 800 and surgical computer, such as a surgical hub, for example. The communications interface 800 may provide data exchange with one or more edge and/or cloud computing resources, for example. Communications interface 824 may receive information such as procedural information, intraoperative reporting information, and the like.

In an example, the communication interface 824 may be used to update the programming of the device 800, including for example, a predictive model used by the device 800. A predictive model may be stored in memory 818, for example. The predictive model may be an updatable predictive model. For example, the communication interface 824, and in turn the processor 816, may receive downloads of the software, firmware, and the like. For a predictive model that includes a neural network. The communication interface 824, and in turn the processor 816, may receive updated coefficients and/or an updated neural network architecture, for example.

The device 800 may have housing and connectors suitable for use in the operating theater. For example, the device 800 may include tubing assemblies and connectors suitable for surgery. The device 800 and/or said connectors may be suitable for an autoclave cycle. For example, the device 800 may be manufactured in accordance with procedures used to provide durable medical equipment. The device 800 may be integrated into a piece of medical equipment typically used in surgery, such as lung resection surgery. The device 800 may be integrated into a ventilator, for example. In an example, the device 800 may be manufactured so that it may be used in surgery to provide an intraoperative indication to the surgeon regarding the likelihood of development of a prolonged air leak.

In an example, the sensors 802, 804, 806, 808, 810, 812 may record various air exchange data. This information may be digitized by way of the analog-to-digital converter 814 at a suitable rate for capturing variations within the breath cycle, such as peaks, variances between peaks, magnitudes, and the like. This signal may be stored in memory 818, processed by the processor 816 and/or the auxiliary processor 820, according to a predictive model, for example. And the user interface 822 may display the output of the predictive model, which may include a probability associated with the likelihood of a development of a prolonged air leak.

In another example, the sensors 802, 804, 806, 808, 810, 812 may record various air exchange data. This information may be digitized by way of the analog-to-digital converter 814 at a suitable rate for capturing variations within the breath cycle, such as peaks, variances between peaks, magnitudes, and the like. This signal may be stored in memory 818, processed by the processor 816, and/or communicated via the communications interface 824. The signals may be paired with one or more other data elements, including patient outcome, such as patient outcome regarding the development of a prolonged air leak. This pairing of signals and outcomes may be used to develop a predictive model for a population.

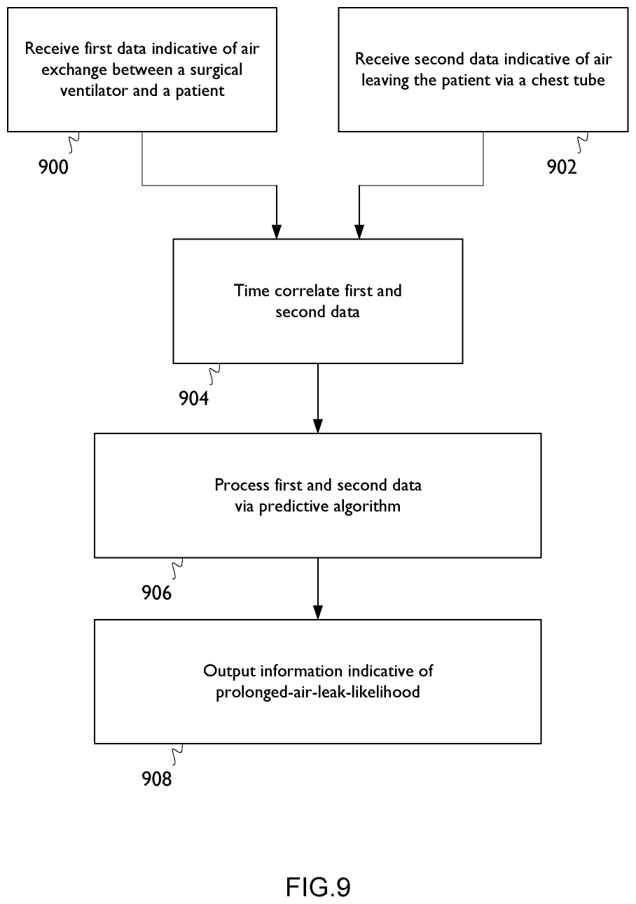

FIG. 9 is a flow diagram of an example process for collecting signals and/or for determining the likelihood of developing a prolonged air leak. In an example, the process may be performed by the device 800 shown in FIG. 8. At 900, first data is received. The data may be indicative of an air exchange between a surgical ventilator and a patient. For example, the first data may be received via a ventilator inlet flow sensor, a ventilator inlet pressure sensor, a ventilator outlet flow sensor, and or a ventilator outlet pressure sensor for example. The first data may include one or more time series. Each time series may be associated with a particular sensor and/or parameter. Such data may be sampled at a rate suitable for capturing variations in the air exchange signal between the surgical ventilator and the patient within a breath cycle, for example.

At 902, second data may be received. The second data may be indicative of air leaving the patient via a chest tube. For example, the second data may include data captured from a chest tube flow sensor, a chest tube pressure sensor, and or the like. The second data may include one or more time series of data. The second data may include a time series associated with each chest tube sensor, for example. Such data may be sampled at a rate suitable for capturing variations in the air leaving the patient via a chest tube, for example.

At 904, the first and second data may be time correlated. For example, the first and second data may be captured in a synchronized fashion. For example, the time correlating of the first and second data may be performed by simultaneous and/or concurrent sampling. For example, the first and second data may include reference to a common clock for purposes of time correlating. For example, the first and second data may be subject to a software-based synchronization. The time correlation of first and second data may be performed by any mechanism or algorithm suitable for capturing time series data such that the various time series may be overlain on a common timeline.

At 906, the first and second data may be processed via a predictive algorithm. The predictive algorithm may include a machine learning algorithm. In an example, the predictive algorithm may be performed by a device, such as device 800 as shown in FIG. 8 for example. In an example, the predictive algorithm may be implemented and or operated on a computing device other than the device collecting the first and second data. For example, the first and second data may be transported to another device by way of a communications interface for purposes of off-board processing.

The output of the predictive algorithm may be outputted, at 908. The output information may be indicative of the likelihood of a prolonged air leak. For example, the output information may include a probability associated with how similar the first and second data are to other first and second data from other populations and their respective patient outcomes.

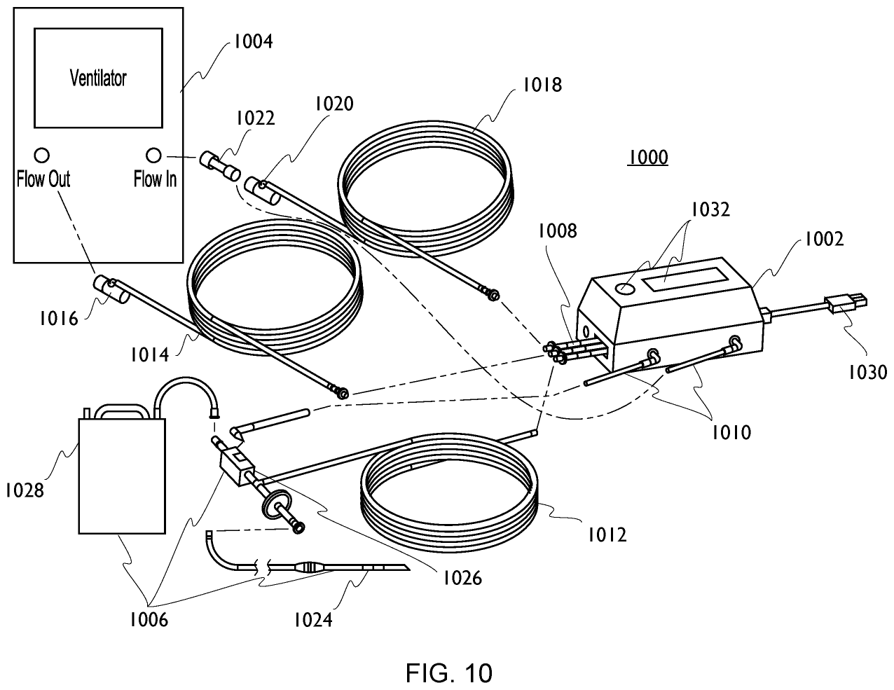

FIG. 10 is an illustration of an example device 1000 for collecting signals and for determining the likelihood of developing a prolonged air leak. The device 1000 may include a console unit 1002. The console unit 1002 may be configured to be connected to a ventilator 1004 and/or a chest tube assembly 1006. The console unit 1002 may include one or more pressure sensor ports 1008. The console unit 1002 may include one or more airflow sensor ports 1010. The console 1002 unit may include data recording hardware and/or software to capture mass airflow and pressure, for example.

One or more pressure sensor ports 1008 may be connected via surgical tubing 1012 to portion of the chest tube assembly 1006 to measure the chest tube pressure. One or more pressure sensor ports 1008 may be connected via surgical tubing 1014 to the outflow port of the ventilator 1004 via a ‘Y’ connection 1016 to measure the outflow ventilator pressure. One or more pressure sensor ports 1008 may be connected via surgical tubing 1018 to the inflow port of the ventilator 1004 via a ‘Y’ connection 1020 to measure the inflow ventilator pressure. Situated between the ‘Y’ connection 1020 and the ventilator inflow port may be an airflow sensor 1022. The airflow sensor 1022 may be connected to an airflow sensor port 1010 of the console unit 1002. The airflow sensor 1022 may be a digital airflow sensor.

The chest tube assembly 1006 may include a chest tube 1024, a chest tube flow sensor 1026, and a water seal drainage unit 1028. The chest tube flow sensor 1026 may be connected between the chest tube 1024 and the water seal drainage unit 1028. For example, the water deal drainage unit may be connected to a vacuum port. The chest tube flow sensor 1026 may be connected to an airflow sensor port 1010 of the console unit 1002 for measuring chest tube airflow. The airflow sensor 1024 may be a digital airflow sensor.

The device 1000 may be suitable to collect signal information from the various sensors before the conclusion of a surgical procedure. For example, the console unit 1002 may obtain flow information from the chest tube flow sensor 1026 and the ventilator airflow sensor 1022. The console unit 1002 may obtain pressure information from the chest tube assembly 1006, the ventilator inport, and the ventilator outport via the one or more pressure sensor ports 108.

The console unit 1002 may record this data in internal memory. The console unit 1002 may include a communications interface connection 1030, such as a USB interface to connect the console unit 1002 to a computer, for example. The console unit 1002 may communicate this data via the communications interface connection 1030 to another computing device.

The console unit 1002 may include a user interface 1032. The user interface 1032 may include one or more buttons and/or displays on the console unit 1002. The console may provide information via the user interface 1032 indicative of the output information from a predictive model. For example, the user interface 1032 may indicate to the surgeon the likelihood of the leak becoming a prolonged air leak, according to a predictive model for example.

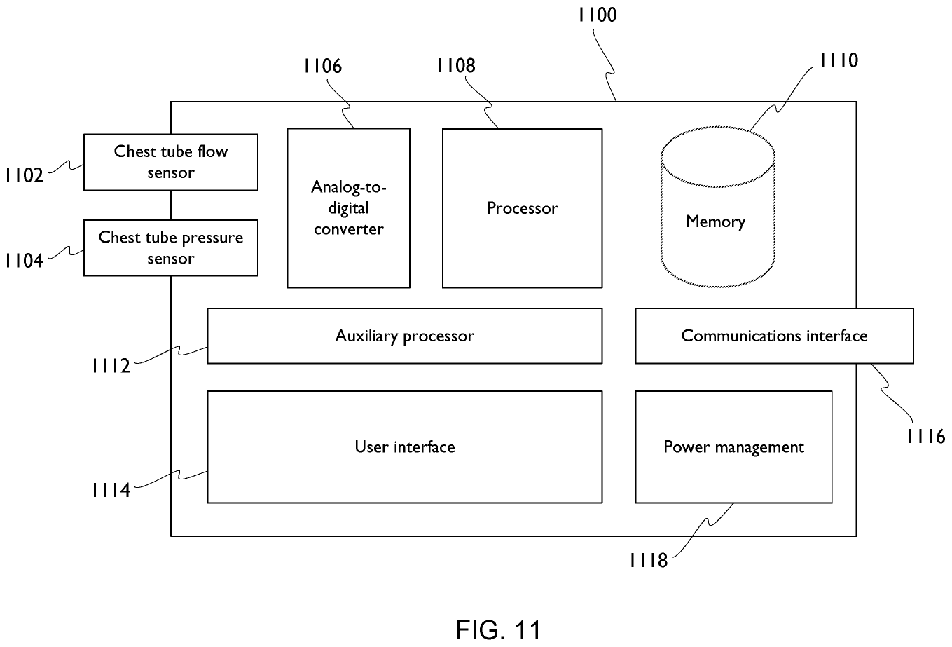

FIG. 11 is a block diagram of an example device 1100 for collecting signals and for determining the likelihood of developing a prolonged air leak. For example, the device 1100 may be suitable for capturing signal information for a population for purposes of training a predictive model. For example, the device 1100 may be suitable for capturing subsequent signal information, such as a testing or new patient signal for example, for purposes of considering the output of a predictive model. For example, the device 1100 may be used to capture subsequent signals to be input to the predictive model and to provide a predicted patient outcome, such as the likelihood of the patient developing a prolonged air leak, to the surgeon.

The device 1100 may include one or more sensors 1102, 1104. The device 1100 may include any sensors suitable for collecting a signal indicative of patient air exchange. For example, the device 800 may include any sensors suitable for collecting a signal indicative of air leaving the patient via a chest tube. For example, the device 1100 may include one or more sensors such as a chest tube flow sensor 1102, a chest tube pressure sensor 1104, and the like. Such sensors may be suitable for recording surgical-quality data from a patient. For example, airflow data may be measured with a tolerance of +/−0.4 liters per minute. For example, pressure measurements may be made with a tolerance of +/−2.5 centimeters H2O.

The flow sensor 1102 may include any sensor suitable for capturing the flow rate of air. For example, the flow sensor 1102 may include sensors or flow meters designed for medical applications. For example, the flow sensor 1102 may be used to withstand autoclave procedures. For example, the flow sensor 1102 may be packaged for single use and/or for a multiple use. For example, the flow sensor 1102 may be designed for medical ventilation or respiratory applications. In an example, the flow sensor 1102 may include an analog sensor and/or digital sensor. For example, the flow sensor 1102 may include one or more silicone sensor chips. For example, the flow sensor 1102 may include relevant support circuitry such as an amplifier, integrated A/D converter, EEPROM memory, digital signaling processing circuitry, and interface circuitry, and the like. In an example, the flow sensor 1102 may include the SFM3400 digital flow meter from Sensirion™.

The pressure sensor 1104 may include any sensor suitable for measuring air pressure. The pressure sensor 1104 may include any sensor suitable for measuring absolute, gauge, and/or differential air pressures, for example. The pressure sensor 1104 may include a sensor or pressure meter designed for medical applications. For example, the pressure sensor 1104 may be used to withstand autoclave procedures. For example, the pressure sensor 1104 may be packaged for single use and/or for a multiple use. For example, the pressure sensor 1104 may include any pressure sensor suitable for medical applications, such as for air monitors, pneumatic controls, respiratory machines, ventilators, spirometers, and the like. In an example, the pressure sensor 1104 may include an analog sensor and/or digital sensor. For example, the pressure sensor 1104 may include one or more silicone sensor chips. For example, the pressure sensor 1104 may include relevant support circuitry such as an amplifier, integrated A/D converter, EEPROM memory, digital signaling processing circuitry, and interface circuitry, and the like. In an example, the pressure sensor 1104 may include a board mount pressure sensor, such as the board mount pressure sensor from Honeywell™ part number 785-HSCDRRN100MD4A3, for example.

Measurements sensed by the sensors 1102, 1104 may be converted to digital representation via one or more analog-to-digital converters 1106. The analog-to-digital converter 1106 may convert an analog representation of the sensor's measurement, such as a voltage, current, or the like, into a digital representation, such as an 8 bit, 16 bit, 24 bit, 32 bit digital value, for example. The analog-to-digital converter 1106 may include any architecture and/or form factor suitable for inclusion in a medical device, such as device 1100. For example, the analog-to-digital converter 1106 may include a converter integrated with one or more sensors themselves. The analog-to-digital converter 1106 may include a subcomponent of the processor 1108. The analog-to-digital converter 1106 may include a standalone electrical component, for example. The analog-to-digital converter 1106 may include an individual converter for each sensor, a shared converter, or a combination thereof.

The analog-to-digital converter 1106 may convert analog information captured by the one or more sensors 1102, 1104 to a digital format by sampling the signals received from the sensors at a particular sampling rate. The sampling rate may be any rate suitable for capturing a signal and/or signal characteristics of air exchange of a patient. For example, the sampling rate may be selected to be at least twice the highest relevant frequency for the type of signal being sampled. For example, the sampling rate may be selected as discussed herein. The digital signals from the analog-to-digital converter 1106 may represent a time series of data for each of the sensors 1102, 1104 of the device 1100. The captured data, for example the one or more time series of data, may be stored in memory 1110 and/or processed by the processor 1108.

The processor 1108 may include any device suitable for processing such data. For example, the processor 1108 may include any device suitable for handling such data, performing numeric operations on such data, storing the data to memory 1110, operating a predictive model with the data as input, handling operation of the device 1100, and/or the like. The processor 1108 may include a general-purpose processor, a microcontroller, an application specific integrated circuit (ASIC), or the like. In an example, the processor may include an Arduino Uno microcontroller, for example.

The memory 1110 may include any component suitable for storing such digital data. For example, the memory 1110 may include random access memory, read-only memory, volatile memory and/or non-volatile memory. For example, the memory 1110 may include a solid-state memory or the like. The memory 1110 may be sized and selected to be suitable for the volume and storage speed required in accordance with the sensors 1102, 1104 and processor 1108.

The device 1100 may include one or more auxiliary processors 1112. An auxiliary processor 1112 may include any component, device, system, computing and/or resource and/or access to such component, device, system, and/or computing resource used to provide processing additional to the processing of processor 1108, for example. The auxiliary processor 1112 may include a Field Programmable Gate Array (FPGA), an application specific integrated circuit (ASIC), an application programming interface (API) to an external processing resource, such as a cloud and/or edge processing resource, and/or the like. In an example, an auxiliary processor may be used to handle the computing requirements of developing and/or implementing a predictive model, such as that discussed herein.

The device 1100 may include a user interface 1114. The user interface 1114 may provide user input mechanisms, such as buttons, touchscreens, and/or access to external user interface devices, such as a keyboard, monitor, and mouse, for example. The user interface 822 may provide a user input mechanism to establish the beginning and/or the end of a signal recording session. The user interface 1114 may provide the ability to input certain patient-related data, for example. The user interface 1114 may provide a user output mechanism, such as indicator lights, a display, access to external user interface devices, and/or the like. The output mechanism may be used to output all or a portion of the information captured by the sensors 1102, 1104. The output mechanism may be used to output a summary of the information captured by the sensors 1102, 1104. The output mechanism may be used to output a result of predictive model processing data captured by the sensors 1102, 1104. For example, the output mechanism may include a likelihood of a prolonged air leak based one data captured from one or more of the sensors 1102, 1104.

The device 1100 may include a communications interface 1116 to provide data exchange between the device 1100 and one or more other components and/or networks. For example, the communication interface 1116 may include a serial interface, a parallel interface, a universal serial bus (USB) interface, and the like. For example, the communication interface 824 may include a network communication interface, such as an Ethernet interface, a WiFi interface, a cellular interface, a 5G interface, and/or the like. The communications interface 1116 may provide access to a host computer, for data logging capabilities, for example. The communications interface 1116 may provide data exchange between the device 1100 and surgical computer, such as a surgical hub, for example. The communications interface 1116 may provide data exchange with one or more edge and/or cloud computing resources, for example. Communications interface 1116 may receive information such as procedural information, intraoperative reporting information, and the like.

In an example, the communication interface 1116 may be used to update the programming of the device 1100, including for example, a predictive model used by the device 1100. A predictive model may be stored in memory 1110, for example. The predictive model may be an updatable predictive model. For example, the communication interface 1116, and in turn the processor 1108, may receive downloads of the software, firmware, and the like. For a predictive model that includes a neural network. The communication interface 1116, and in turn the processor 1108, may receive updated coefficients and/or an updated neural network architecture, for example.

The device 1100 may have housing and connectors suitable for use in the operating theater. For example, the device 1100 may include tubing assemblies and connectors suitable for surgery. The device 1100 and/or said connectors may be suitable for an autoclave cycle. For example, the device 1100 may be manufactured in accordance with procedures used to provide durable medical equipment. The device 1100 may be integrated into a piece of medical equipment typically used in surgery, such as lung resection surgery. The device 1100 may be integrated into a ventilator, for example. In an example, the device 1100 may be manufactured so that it may be used in surgery to provide an intraoperative indication to the surgeon regarding the likelihood of development of a prolonged air leak.

The device 1100 may include a power management subsystem 1118. The power management subsystem 1118 may include an onboard power source, such as a battery for example. The power source and power management subsystem 1118 may enable the device 1100 to be a portable device. Such a portable device may be sent home with a patient to continuously and/or intermittently collect information via the sensors 1102, 1104 and communicate this information via communications interface 1116, such as a wireless cellular interface for example. The power management subsystem 1118 may provide power management for the device 1100, including scheduled measurement-taking between periods of a low power “sleep” mode. The power management subsystem 1118 may enable device 1100, in a portable setting, to provide days accurate signal recordings and provide those recordings to a health care professional.

In an example, the sensors 1102, 1104 may record various air exchange data. This information may be digitized by way of the analog-to-digital converter 1106 at a suitable rate for capturing variations within the breath cycle, such as peaks, variances between peaks, magnitudes, and the like. This signal may be stored in memory 1110, processed by the processor 1108 and/or the auxiliary processor 1112, according to a predictive model, for example. And the user interface 1114 may display the output of the predictive model, which may include a probability associated with the likelihood of a development of a prolonged air leak.

In another example, the sensors 1102, 1104 may record various air exchange data. This information may be digitized by way of the analog-to-digital converter 1106 at a suitable rate for capturing variations within the breath cycle, such as peaks, variances between peaks, magnitudes, and the like. This signal may be stored in memory 1110, processed by the processor 1108, and/or communicated via the communications interface 1116. The signals may be paired with one or more other data elements, including patient outcome, such as patient outcome regarding the development of a prolonged air leak. This pairing of signals and outcomes may be used to develop a predictive model for a population.

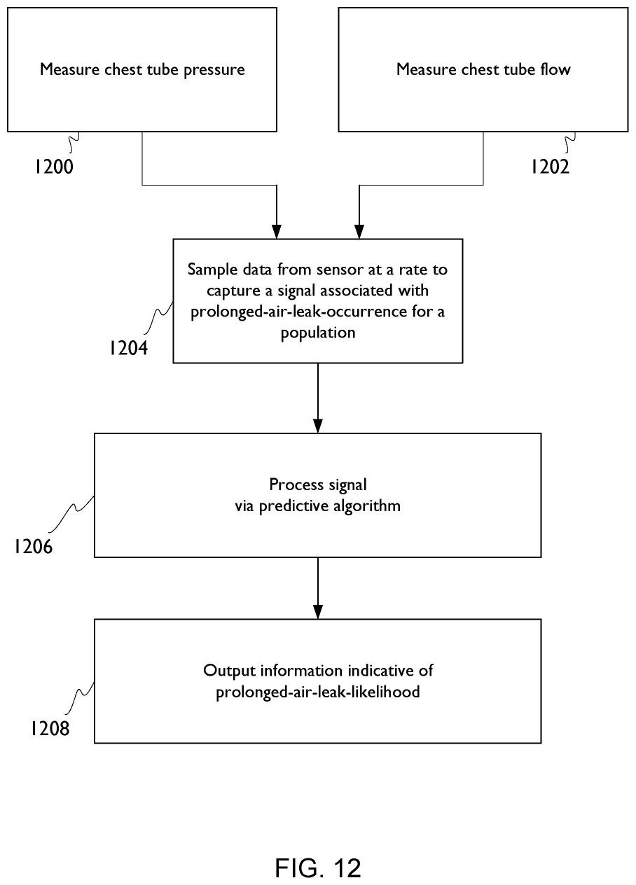

FIG. 12 is a flow diagram of an example process for collecting signals and for determining the likelihood of developing a prolonged air leak. For example, this process may be employed by device 1100.

At 1200, chest tube pressure may be measured. For example, the chest tube pressure may be measured by a pressure sensor. At 1202, chest tube flow rate may be measured. For example, chest tube flow rate may be measured by an air mass flow sensor. Chest tube pressure and chest tube flow may be measured by respective analog sensors with an analog-to-digital converter. They may be measured by digital sensors with integrated analog-to-digital converters.

At 1204, data may be sampled from the respective sensors at a suitable for capturing a signal associated with prolonged air leak occurrence for a population. For example, the signal may be sampled at a rate at least twice the highest relevant frequency for the sensors and/or the signal being captured. For example, the rate may be suitable for capturing the pressure and flow variances within a particular or individual breath cycle.

At 1206, the signal may be processed according to a predictive algorithm. The predictive algorithm may include a machine learning algorithm trained based on chest tube pressure and chest tube flow and patient outcomes. And 1208, information indicative of a prolonged air leak likelihood may be output to the user. For example, information indicative of prolonged-air-leak-likelihood may be displayed to a user. In an example, an adjustment to post-operative care may be determined based on the information indicative of prolonged-air-leak-likelihood. In an example, an adjustment to intra-operative care may be determined based on the information indicative of prolonged-air-leak-likelihood. In an example, a risk level for at least one surgical outcome may be determined based, at least in part, on the information indicative of prolonged-air-leak-likelihood. For example, the risk level may be compared to a predetermined threshold. The predetermined threshold may be predetermined according to historical data related to the particular surgery, clinical trials, expert opinion, and the like.

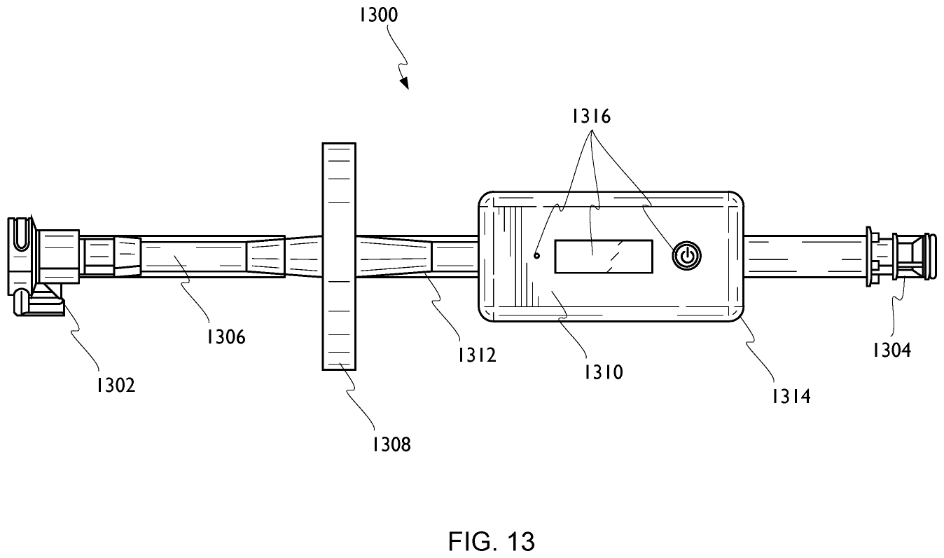

FIG. 13 is an illustration of an example device 1300 for collecting signals and for determining the likelihood of developing a prolonged air leak. The device 1300 may be used in connection with a chest tube and drainage system, for example. For example, the device 1300 may be situated between a chest tube and a chest tube drain tubing, for example. The device may include a first connection 1302. The first connection 1302 may connect to the chest tube. The first connection 1302 may connect to the chest tube by way of a quick-connect fitting, for example. The device may include a second connection 1304. The second connection 1304 may connect to the chest tube drainage unit. The second connection 1304 may connect to the chest tube drainage unit via a quick-connect fitting, for example.

The device 1300 may include a tube 1306 between the first connection 1302 and a first side of a filter 1308. The filter 1308 may include a second side opposite the first side. The device 1300 may include a main module 1310. And a tube 1312 may connect an input of the main module 1310 to the second side of the filter 1308. The main module 1310 may have an output. The output may be connected to the second connection 1304.

The main module 1310 may include an integrated system for collecting signals and for determining the likelihood of developing a prolonged air leak. The main module 1310 may include a housing 1314. Inside the housing 1214 may be components such as a wireless communications module, a pressure transducer, a flow meter, a memory, an EEPROM, a processor, and/or an integrated battery. The device 1300 may include, for example, an integrated implementation of device 1100 shown in FIG. 11. The main module 1310 may have one or more user interface elements 1316. For example, the user interface elements 1316 may elements such as a power switch, an indicator, a display, and/or an audible alarm, for example.

In use, the device 1300 may be connected between a patient's chest tube and corresponding drain tubing. And the device 1300 may employ the process shown in FIG. 12, for example. In an example, the device 1300 may be connected to the patient's chest tube and corresponding drain tubing before the patient's surgery is complete. The device 1300 may be operated by turning on the power switch. In an example, configuration information may be provided to the device 1300 by way of a wireless communication module. The device 1300 may collect pressure and flow signal information. In an example, the device 1300 may include an onboard predictive model for processing. In an example, the device 1300 may capture and/or receive information and send it via a wireless communications module to an external computing device, such as a surgical hub, edge server, cloud computing system, or the like. And the device 1300 may display, via a user interface element, a predicted probability of the development of a prolonged air leak based on the collected information.

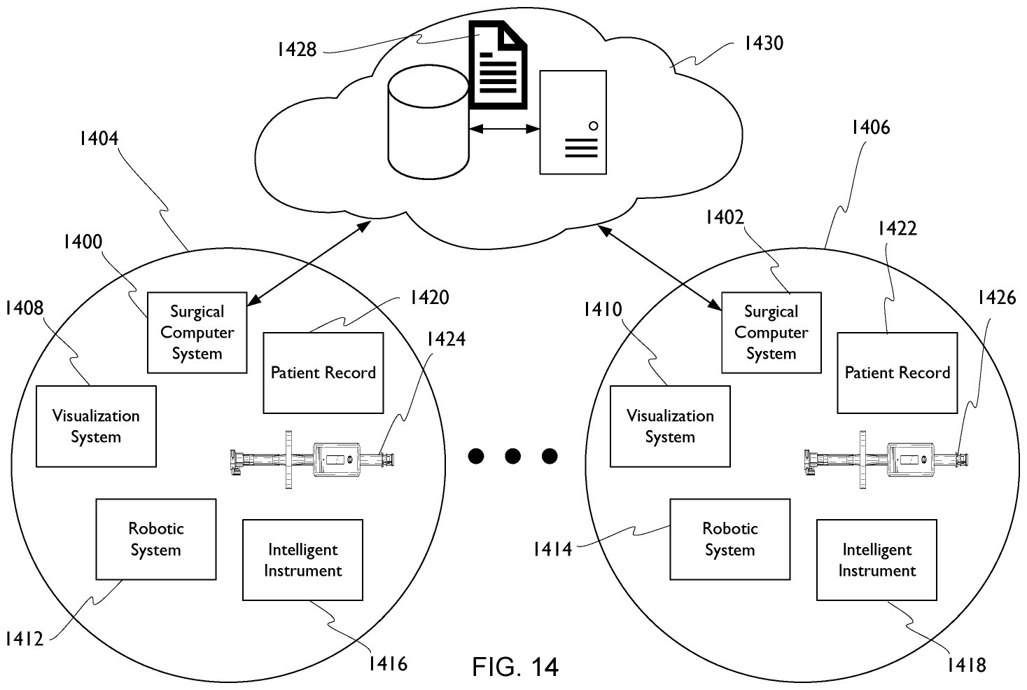

FIG. 14 is a block diagram of a system incorporating an example plurality of surgical computing devices 1400, 1402. The devices 1400, 1402 may be located in separate locations 1404, 1406. The locations 1404, 1406 may be separated physically and/or logically, such as different operating rooms in the same hospital, in different hospitals in the same health care system, in different health care systems with a common data network, or the like.

The surgical computing device 1400, 1402 may be used in an operating room. In the operating room, the surgical computing device 1400, 1402 may coordinate and/or aggregate data and/or control of surgical equipment. For example, the surgical computing device 1400, 1402 may coordinate and/or aggregate data associated with surgical equipment such as a surgical visualization system 1408, 1410, a surgical robot system 1412, 1414, one or more intelligent instruments 1416, 1418, patient record systems 1420, 1422, air exchange monitoring devices 1424, 1426 (e.g., as disclosed herein), and the like. In an example, the surgical computing device 1400, 1402 may coordinate and/or aggregate data associated with surgical equipment such as imaging devices, illumination sources, displays, intelligent surgical instruments, patient monitoring equipment, anesthesia machine surgical table, surgical microscope, vital signs monitor, EKG machine, ultrasound machine, endoscopy equipment, electrosurgical devices, surgical staplers, smoke evacuator, nerve stimulator, central gas and suction controls, and the like. Such devices may generate and/or consume data feeds associated with the surgical procedure.

In an example, the surgical computing device 1400, 1402 may include one or more modules for use in a surgical procedure. For example, the surgical computing device 1400, 1402 may include a monitor, an imaging module, a generator module, a smoke evacuation module, a suction/irrigation module, a communications module, a processor module, a storage array, an operating room mapping module, and the like. The combo generator module may include two or more of an ultrasonic energy generator component, a bipolar RF energy generator component, and a monopolar RF energy generator component that are housed in a single unit. Such modules may be included in a modular enclosure.

The modules and surgical equipment may generate and/or consume data feeds relevant to the surgical procedure. Such data feeds may represent information about the surgical procedure, such as patient medical record information, pre-surgical planning, intraoperative activities, including equipment usage, surgical tasks, patient response, patient outcome, and the like. And, the incorporation of a surgical computing device 1400, 1402 in a surgical setting may enable the collection of this relevant surgical data 1428. For example, the surgical data 1428 may include data such as patient medical record data, inoperative reporting data, surgical procedure data, and the like.

Such devices may interact with the surgical computing device 1400, 1402. The surgical computing device 1400, 1402 may include a surgical hub. For example, the surgical computing device 1400, 1402 and corresponding surgical data 1428 may include that disclosed in the following, which are incorporated by reference herein:
U.S. patent application Ser. No. 15/640,656, (Attorney Docket No. END8499USNP2) entitled “SURGICAL HUB COORDINATION OF CONTROL AND COMMUNICATION OF OPERATING ROOM DEVICES,” filed on Mar. 29, 2018, (U.S. Patent Application Publication No. 2019-0201141 A1)
U.S. patent application Ser. No. 15/940,668, (Attorney Docket No. END9502USNP2) entitled “AGGREGATION AND REPORTING OF SURGICAL HUB DATA,” filed on Mar. 29, 2018, (U.S. Patent Application Publication No. 2019-0201115 A1)
U.S. patent application Ser. No. 16/209,385, (Attorney Docket No. END9495USNP) entitled “METHOD OF HUB COMMUNICATION, PROCESSING, STORAGE AND DISPLAY,” filed on Dec. 4, 2018, (U.S. Patent Application Publication No. US 2019-0200844 A1)
U.S. patent application Ser. No. 16/209,407, (Attorney Docket No. END8497USNP) entitled “METHOD OF ROBOTIC HUB COMMUNICATION, DETECTION, AND CONTROL,” filed on Dec. 4, 2018, (U.S. Patent Application Publication No. US 2019-0201137 A1)
U.S. patent application Ser. No. 16/209,403, (Attorney Docket No. END8496USNP) entitled “METHOD OF CLOUD BASED DATA ANALYTICS FOR USE WITH THE HUB,” filed on Dec. 4, 2018, (U.S. Patent Application Publication No. US 2019-0206569 A1)
U.S. patent application Ser. No. 16/209,416, (Attorney Docket No. END8505USNP) entitled “METHOD OF HUB COMMUNICATION, PROCESSING, DISPLAY, AND CLOUD ANALYTICS,” filed on Dec. 4, 2018, (U.S. Patent Application Publication No. US 2019-0206562 A1)
U.S. patent application Ser. No. 15/940,671, (Attorney Docket No. END8502USNP) entitled “SURGICAL HUB SPATIAL AWARENESS TO DETERMINE DEVICES IN OPERATING THEATER,” filed on Mar. 29, 2018, (U.S. Patent Application Publication No. US 2019-0201104 A1)
U.S. patent application Ser. No. 15/940,654, (Attorney Docket No. END8501USNP) entitled “SURGICAL HUB SPATIAL AWARENESS TO DETERMINE DEVICES IN OPERATING THEATER,” filed on Mar. 29, 2018, (U.S. Patent Application Publication No. US 2019-0201140 A1)
U.S. patent application Ser. No. 16/209,478, (Attorney Docket No. END9015USNP1) entitled “METHOD FOR SITUATIONAL AWARENESS FOR SURGICAL NETWORK OR SURGICAL NETWORK CONNECTED DEVICE CAPABLE OF ADJUSTING FUNCTION BASED ON A SENSED SITUATION OR USAGE,” filed on Dec. 4, 2018, (U.S. Patent Application Publication No. US 2019-0104919 A1)
U.S. patent application Ser. No. 16/209,490, (Attorney Docket No. END9017USNP1) entitled “METHOD FOR FACILITY DATA COLLECTION AND INTERPRETATION,” filed Dec. 4, 2018, (U.S. Patent Application Publication No. 2019-0206564 A1)
U.S. patent application Ser. No. 17/358,270, (Attorney Docket No. END8505USCNT2) entitled “METHOD OF HUB COMMUNICATION, PROCESSING, DISPLAY, AND CLOUD ANALYTICS,” filed Jun. 25, 2021, (U.S. Patent Application Publication No. 2021-0315580 A1)
U.S. patent application Ser. No. 17/210,839, (Attorney Docket No. END9018USCNT1) entitled “SURGICAL NETWORK RECOMMENDATIONS FROM REAL TIME ANALYSIS OF PROCEDURE VARIABLES AGAINST A BASELINE HIGHLIGHTING DIFFERENCES FROM THE OPTIMAL SOLUTION,” filed Mar. 24, 2021, (U.S. Patent Application Publication No. 2021-0205020 A1)
U.S. patent application Ser. No. 17/217,163, (Attorney Docket No. END8500USCNT1) entitled “DATA STRIPPING METHOD TO INTERROGATE PATIENT RECORDS AND CREATE ANONYMIZED RECORD,” filed Mar. 30, 2021, (U.S. Patent Application Publication No. 2021-0240852 A1)
U.S. patent application Ser. No. 17/363,944, (Attorney Docket No. END8495USCNT2) entitled “METHOD OF HUB COMMUNICATION,” filed Jun. 30, 2021, (U.S. Patent Application Publication No. 2021-0322018 A1)
U.S. patent application Ser. No. 16/182,278, (Attorney Docket No. END9018USNP4) entitled “COMMUNICATION OF DATA WHERE A SURGICAL NETWORK IS USING CONTEXT OF THE DATA AND REQUIREMENTS OF A RECEIVING SYSTEM/USER TO INFLUENCE INCLUSION OR LINKAGE OF DATA AND METADATA TO ESTABLISH CONTINUITY,” filed Nov. 6, 2018, (U.S. Patent Application Publication No. 2019-0201130 A1)
U.S. patent application Ser. No. 16/182,260, (Attorney Docket No. END9012USNP8) entitled “AUTOMATED DATA SCALING, ALIGNMENT, AND ORGANIZING BASED ON PREDEFINED PARAMETERS WITHIN SURGICAL NETWORKS,” filed Nov. 6, 2018, (U.S. Pat. No. 11,056,244)
U.S. patent application Ser. No. 16/172,198, (Attorney Docket No. END8571USNP) entitled “METHOD OF HUB COMMUNICATION WITH SURGICAL INSTRUMENT SYSTEMS,” filed Oct. 26, 2018, (U.S. Patent Application Publication No. 2019-0125455 A1)
U.S. patent application Ser. No. 15/940,649, (Attorney Docket No. END8500USNP3) entitled “DATA PAIRING TO INTERCONNECT A DEVICE MEASURED PARAMETER WITH AN OUTCOME,” filed Mar. 29, 2018, (U.S. Patent Application Publication No. 2019-0205567 A1)
U.S. patent application Ser. No. 16/931,712, (Attorney Docket No. END9032USCNT1) entitled “WIRELESS PAIRING OF A SURGICAL DEVICE WITH ANOTHER DEVICE WITHIN A STERILE SURGICAL FIELD BASED ON THE USAGE AND SITUATIONAL AWARENESS OF DEVICES,” filed Jul. 17, 2020, (U.S. Pat. No. 11,179,204)
U.S. patent application Ser. No. 16/182,256, (Attorney Docket No. END9016USNP2) entitled “ADJUSTMENT OF A SURGICAL DEVICE FUNCTION BASED ON SITUATIONAL AWARENESS,” filed Nov. 6, 2018, (U.S. Patent Application Publication No. 2019-0201127 A1)

In an example, such surgical data 1428 may be stored at a storage array in at the surgical computing device 1400, 1402. In an example, such surgical data 1428 may be aggregated from one or more surgical computing devices 1400, 1402 at a data repository 1430. Such surgical data 1428 may be used to develop the predictive model.

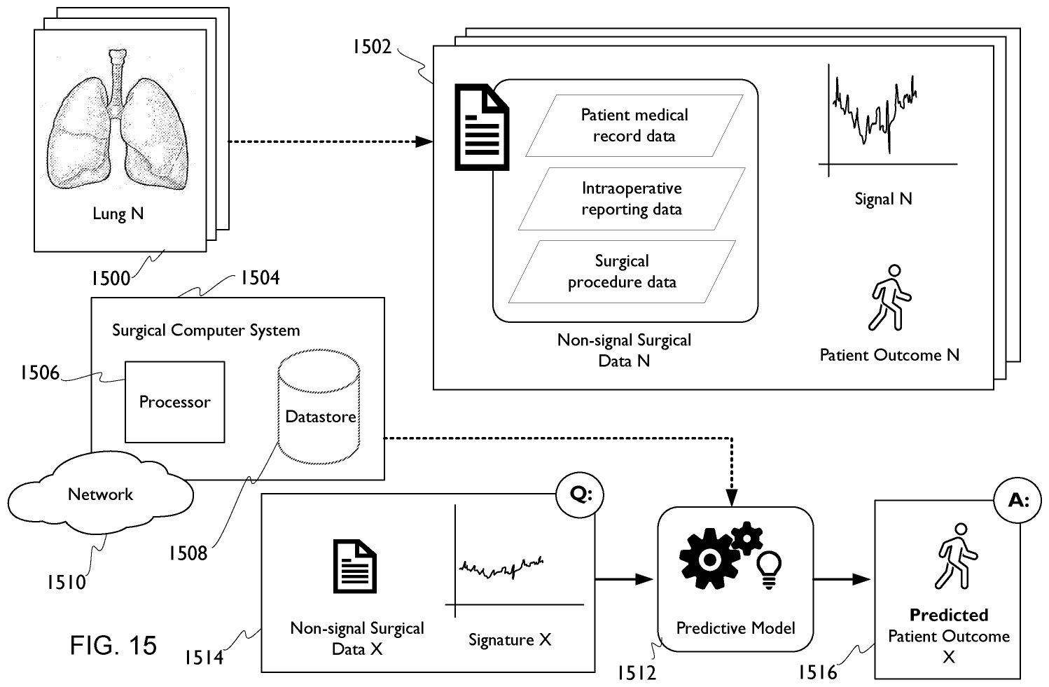

FIG. 15 is a block diagram illustrating an example system for determining the likelihood of developing a prolonged air leak. Surgical data 1500 may be captured from one or more resection surgeries. For example, surgical data 1500 may be captured from a large number of resection surgeries. For example, the data may be collected from at least a hundred resection surgeries. The surgical data 1500 may comprise one or more signals indicative of the air exchange of the respective patient. The surgical data 1500 may include non-signal surgical data, such as that collected and/or aggregated from one or more surgical computing devices, such as the surgical computing devices 1400, 1402, as shown in FIG. 14, for example.

The surgical data 1500 may be prepared into data elements 1502. The data elements 1502 may include at least a signal and/or signal characteristics, a corresponding patient outcome, and one or more parameters of corresponding non-signal surgical data.

In an example, the non-signal surgical data may include data such as patient medical record data, inoperative reporting data, surgical procedure data, and the like. For example, the patient medical record data may include any data associated with the patient and/or his or her medical treatment. For example, the patient record data may include identification information, such as date of birth, name, marital status, social security number, and the like, patient number, patient identifier. Patient medical record data may include medical history, such as allergies, previous treatment, previous medical care, present and past diagnoses. The patient medical record data may include medication information, which is a record of medicines that the patient is currently ingesting. The medication information may include prescribed medicines as well as non-prescribed medicines such as herbal remedies, illegal substances, over the counter medication, and the like.

The patient medical record data may include information regarding demographic information, race, ethnicity, family history, and the like. For example, family history may include family history related to lung. The patient medical record data may include information regarding treatment history. The treatment history may reflect information regarding treatments that the patient has undergone and/or their results. For example, the treatment history information may include chief complaints, history of illness, vital signs, physical examination remarks, surgical history, obstetric history, allergies, family history, immunization history, habits, including diet, alcohol intake, exercise, drug abuse, smoking, developmental history, age, weight, sex, and the like.

The patient's medical record data may include lab results. The lab results may include, lab results related to cells, tissues, and/or bodily fluids, for example. Imaging records such as x-ray, CT scans, ultrasounds may be included in such lab results. The patient medical record data may include progress notes. For example, the progress notes may indicate specific information during the course of treatment, during surgery, and/or during recovery from that surgery. For example, the progress notes may include information related to bowel and bladder functions, observation of the mental and physical condition of the patient, sudden changes in physiology or behavior, food intake, and/or vital signs. For example, the progress notes may include information related to air leak metrics, such as chest tube air mass metrics and the like. In an example, the patient medical record data may include preoperative data and/or the results of one or more tests. For example, patient spirometry, diffusion capacity, myocardial stress test, echocardiogram, quantitative perfusion scanning, imaging, specific landmarks of unusual anatomy and lesion location for example, the presence of chemotherapy, radiation, smoking, COPD, and the like.

The patient medical record data may include information related to postoperative information, such as patient mobilization, respiratory exercises, the use of an incentive spirometer, the chest tube draining rate, the overall chest tube liquid draining rate, whether the chest tube is operated with a vacuum or a water seal, and the use of a portable pleural vac, and the like.

Intraoperative reporting data may include any information relating to the performance of the operative procedure. For example, such intraoperative reporting data may be collected automatically by way of a surgical hub for example. Intraoperative reporting data may be captured as notes from the surgeon and/or surgical nurse. Intraoperative reporting data may include information such as tumor locations and adhesions. Intraoperative reporting data may include patient positioning and/or changes in the patient position during surgery, for example. Intraoperative reporting may include information regarding the general progress of the procedure. For example, the intraoperative reporting may include whether a lung resection surgery was performed as an open surgery or VATS. The intraoperative reporting may include whether a lung resection surgery was initiated as VATS but was converted to open during the surgery. In an example, the intraoperative reporting may include VATS port site placements.

The reporting may include information regarding wound closure, such as how wounds in the lung were closed, their dimensions, locations, and the like, for example. The intraoperative reporting data may indicate the surgical stapler used, staple type, the number of staple firings, pressure level, firing timing, and the like. To the extent sutures are used, the inoperative reporting data may include number of sutures, size of the incision being sutured, suture type, and the like. With respect to procedural intraoperative reporting data to the extent that there's a bronchial stump, the inoperative reporting data may include whether or not the bronchial stump is covered with a well vascularized tissue flap, such as that from the pericardial fatty tissue, pleural flap, intercoastal muscle flap, or pedicle diaphragm flap, and the like.

The intraoperative reporting data may include information relating to tissue separation, such as electrocautery, bipolar current, ultracision harmonic scalpel use, and the like. The reporting data may include information about such tools including, for example, instrument type, instrument settings, and use quantity and/or duration, and the like. The reporting may include information on bleeding, such as amount of bleeding, the nature of bleeding, method of coagulation, and the like, for example. The intraoperative reporting data may include visual imaging to collect situational awareness around the position, location, and/or use of incision tools and sealants. The intraoperative reporting data may include the lung pressure at a specific time of sealing.

Other inoperative reporting data may include whether air leak is detected by inflation of the lung underwater, the number and placement of chest tubes for example, the presence and/or treatment of solitary leakages, the use of laser, such as a CO2 laser for ceiling air leaks.

The surgical procedure data may include any information related to the planned procedure. This may include the nature of the surgery, whether it was trauma-related, cancer-related, or the like. To the extent that the surgical procedure is a lung resection, the surgical procedure data may include the magnitude of the procedure, such as whether the procedure includes a pneumonectomy, a lobectomy, a sublobectomy resection, and the like. For example, in a lobectomy, the procedure data may include information such as, a right upper lobe procedure performed open, a right upper lobe procedure performed by way of VATS, an open middle lobectomy, a right middle lobe procedure by way of VATS, a right middle lobe procedure performed open, a right lower lobe procedure by way of VATS, a right lower lobe procedure performed open, a left upper lobe procedure by way of VATS, a left upper lobe procedure performed open, and/or the like.

Intraoperative data may include other information such as which instruments are being used, how long the surgery is being done, recorded data from any visual recording, supplemental intraoperative imaging recording, and/or the recording of data from one or more wearable devices, such as those worn by the healthcare professionals during the procedure, and the like.

The surgical data 1500 and/or the data elements 1502 may be stored and processed by a surgical computer system 1504. The surgical computer system 1504 may include a processor 1506 and/or a datastore 1508. The surgical computer system 1504 may be operable over a computer network 1510. The surgical computer system 1504 may be located within a Health Insurance Portability and Accountability Act (HIPAA) boundary or outside a HIPPA boundary. The collected data may populate the datastore 1508 such that a predictive model 1512 may be generated. The processor 1506 may be configured to generate the predictive model 1512 based on the surgical data 1500 and/or the data elements 1502 stored in the datastore 1508, for example. The predictive model 1512 may be generated by traditional data analysis techniques, machine learning, and the like. The predictive model 1512 may be generated by the processor 1506 in accordance with any appropriate machine learning technique, such as those discussed above.

In an example, the predictive model 1514 may be generated, validated, and ultimately deployed. For example, an input 1514 may include a subsequent signal and corresponding subsequent non-signal surgical data. This input 1514 may be input to the predictive model 1512 to provide an output 1516. The output 1516 may include a probability of whether the subsequent signal and subsequent non-signal surgical data is associated with the development of a prolonged air leak. The inclusion of non-signal surgical data as input to the model may improve the model's predictive capacity for a population, for example.

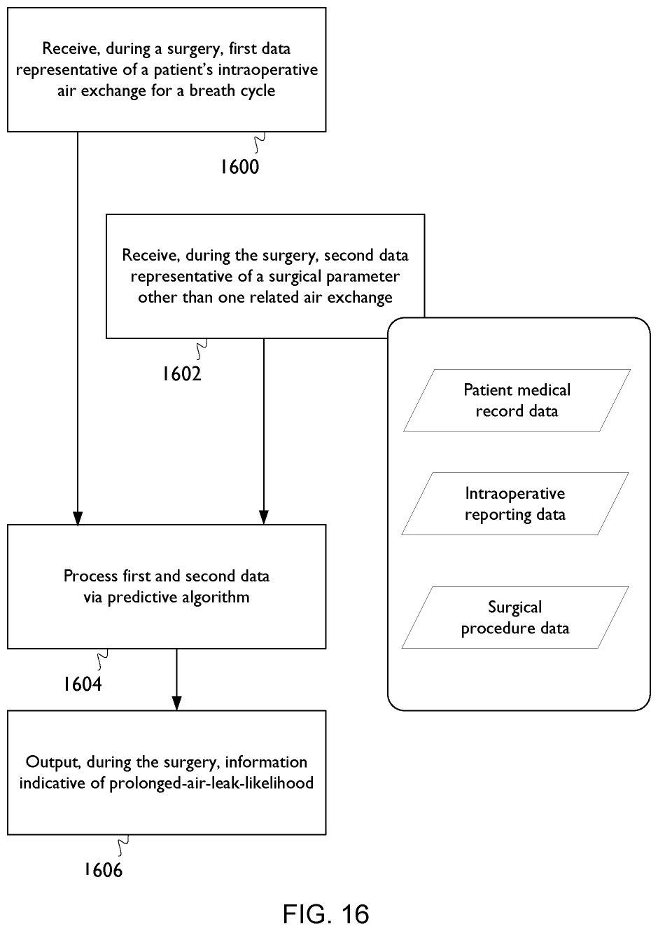

FIG. 16 is a flow diagram of an example process for collecting signals and for determining the likelihood of developing a prolonged air leak.

At 1600, during a surgery, first data may be received. This first data may be representative of a patient's intraoperative air exchange for a given breath cycle. For example, the first data may be collected by a device such as that in FIG. 8 and/or FIG. 11 for example. The first data may include a signal.

At 1602, also during the surgery, second data may be received. The second data may be representative of a surgical parameter other than one related to air exchange. For example, the second data may include non-signal surgical data. For example, the second data may include data such as patient medical record data, inoperative reporting data, surgical procedure data, and the like. Here, second data, related to the surgery, may be used to further enhance the effectiveness of the predictive algorithm.

At 1604, the first and second data may be processed. The first and second data may be processed via predictive algorithm for example. And, at 1606, during the surgery, information indicative of a prolonged air leak likelihood may be output to the surgeon. For example, information indicative of prolonged-air-leak-likelihood may be displayed to a user. In an example, an adjustment to post-operative care may be determined based on the information indicative of prolonged-air-leak-likelihood. In an example, an adjustment to intra-operative care may be determined based on the information indicative of prolonged-air-leak-likelihood. In an example, a risk level for at least one surgical outcome may be determined based, at least in part, on the information indicative of prolonged-air-leak-likelihood. For example, the risk level may be compared to a predetermined threshold. The predetermined threshold may be predetermined according to historical data related to the particular surgery, clinical trials, expert opinion, and the like.

In an example, a treatment recommendation may be generated and/or output. The treatment recommendation may be based, at least in part, on the information indicative of prolonged-air-leak-likelihood, for example. The treatment recommendation may be intended to improve surgical outcome. The treatment recommendation may be based on according to historical data related to the particular surgery, clinical trials, expert opinion, and the like, for example.

1. A device comprising:
a processor configured to:
receive, during a surgery, first data, said first data being representative of a patient's intraoperative air exchange for a breath cycle,
receive, during the surgery, second data, said second data being representative of a surgical parameter other than one related air exchange, and
output, during the surgery, information indicative of prolonged-air-leak-likelihood based on the first and second data.

2. The device of claim 1, wherein the first data comprises any of ventilator inlet flow rate, ventilator inlet pressure, ventilator output pressure, chest tube flow rate, or chest tube pressure.

3. The device of claim 1, wherein the second data comprises patient medical record data.

4. The device of claim 1, wherein the second data comprises intraoperative reporting data.

5. The device of claim 1, wherein the second data comprises procedure data associated with the surgery.

6. The device of claim 5, wherein the procedure data comprises information characterizing a type of lung resection being performed during the surgery.

7. The device of claim 1, wherein the information indicative of prolonged-air-leak-likelihood is based on a machine learning model to which the first data and second data are input.

8. The device of claim 1, wherein the processor is configured to receive an updated machine learning model from a cloud resource and wherein the information indicative of prolonged-air-leak-likelihood is based on the updated machine learning model to which the first data and second data are input.

9. A method comprising:
receiving, during a surgery, first data, said first data being representative of a patient's intraoperative air exchange for a breath cycle,
receiving, during the surgery, second data, said second data being representative of a surgical parameter other than one related air exchange, and
outputting, during the surgery, information indicative of prolonged-air-leak-likelihood based on the first and second data.

10. The method of claim 9, wherein the first data comprises any of ventilator inlet flow rate, ventilator inlet pressure, ventilator output pressure, chest tube flow rate, or chest tube pressure.

11. The method of claim 9, wherein the second data comprises patient medical record data.

12. The method of claim 9, wherein the second data comprises intraoperative reporting data.

13. The method of claim 9, wherein the second data comprises procedure data associated with the surgery.

14. The method of claim 13, wherein the procedure data comprises information characterizing a type of lung resection being performed during the surgery.

15. The method of claim 9, wherein the information indicative of prolonged-air-leak-likelihood is based on a machine learning model to which the first data and second data are input.

16. The method of claim 9, further comprising receiving an updated machine learning model from a cloud resource, wherein the information indicative of prolonged-air-leak-likelihood is based on the updated machine learning model to which the first data and second data are input.

17. The method of claim 9, further comprising displaying the information indicative of prolonged-air-leak-likelihood.

18. The method of claim 9, further comprising determining an adjustment to post-operative care based on the information indicative of prolonged-air-leak-likelihood.

19. The method of claim 9, further comprising determining an adjustment to intra-operative care based on the information indicative of prolonged-air-leak-likelihood.

20. The method of claim 9, further comprising determining a risk level for at least one surgical outcome based, at least in part, on the information indicative of prolonged-air-leak-likelihood.

21. The method of claim 20, further comprising comparing the risk level to a predetermined threshold.

22. A device comprising:
a computer configured to:
receive first data being representative of a plurality of intraoperative air exchanges respective to individual patients in a population,
receive second data being representative of respective patient outcomes corresponding to the individual patients in the population,
receive third data being representative of a respective surgical parameter other air exchange and outcome corresponding individual patients in a population,
to update a model of prolonged-air-leak-likelihood based on the first, second, and third data, and
send the model to a surgical hub, wherein the surgical hub is configured to:
receive, during a surgery, fourth data, said fourth data being representative of a patient's intraoperative air exchange for a breath cycle,
receive, during the surgery, fifth data, said second data being representative of a surgical parameter other than one related air exchange,
output, during the surgery, information indicative of prolonged-air-leak-likelihood based on the fourth data, the fifth data, and the model.

23. The device of claim 22, wherein the third data comprises patient medical record data.

24. The device of claim 22, wherein the third data comprises intraoperative reporting data.

25. The device of claim 22, wherein the third data comprises procedure data corresponding to respective surgical procedures of the respective intraoperative air exchanges.

26. The device of claim 22, wherein the surgical hub is further configured to generate a treatment recommendation based, at least in part, on the information indicative of prolonged-air-leak-likelihood, wherein the treatment recommendation is intended to improve a surgical outcome.
